(19) United States
(12) Patent Application Publication
(10) Pub. No.: US 2006/0099191 A1
(43) Pub. Date: May 11, 2006

(54) Cell-seeded tissue-engineered polymers for treatment of intracranial aneurysms

(75) Inventors: Brian L. Hoh, Charlestown, MA (US); Christopher S. Ogilvy, Winchester, MA (US); Johnny C. Pryor, Lexington, MA (US); Joseph P. Vacanti, Winchester, MA (US)

Correspondence Address: EDWARDS & ANGELL, LLP, P.O. BOX 55874, BOSTON, MA, 02205, US

(73) Assignee: The General Hospital Corporation, Boston, MA (US)

(21) Appl. No.: 10/531,544

(22) PCT Filed: Oct. 16, 2003
(86) PCT No.: PCT/US03/32593

§ 371 Date: Sep. 26, 2005

Related U.S. Application Data
(60) Provisional application No. 60/419,052, filed on Oct. 16, 2002.

(51) Int. Cl. A61M 31/00; A61K 35/44

(52) U.S. Cl. 424/93.7; 604/500

(57) ABSTRACT

Methods and materials for treating intracranial aneurysms with a tissue-engineered biopolymer are disclosed. A novel technique has been developed which utilizes a tissue-engineering biopolymer (TEBP) with living endothelial cells such that the endothelial cells will produce a neoendothelium across the aneurysm ostium. Alternatively, biocompatible materials can be coated onto aneurysm maintenance devices, and the cells can be seeded onto the biocompatible material.

### CROSS REFERENCE TO RELATED APPLICATIONS

This application claims priority to U.S. Provisional Application Ser. No. 60/419,052, filed Oct. 16, 2002.

Reference is made to the following related applications: U.S. patent application Ser. No. 10/150,828, filed May 16, 2002, Ser. No. 10/187,247 filed Jun. 28, 2002, and Ser. No. 09/560,480 filed Apr. 28, 2000; and International Patent Application Numbers PCT/US02/15813, filed May 16, 2002, and PCT/US00/11407, filed Apr. 28, 2002 and published as WO 00/66036 on Nov. 9, 2000.

Each of the foregoing applications and patents, each foregoing publication, and each document cited or referenced in each of the foregoing applications and patents, including during the prosecution of each of the foregoing applications and patents (“application and article cited documents”), and any manufacturer's instructions or catalogues for any products cited or mentioned in each of the foregoing applications and patents and articles and in any of the application and article cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

### FIELD OF THE INVENTION

The present invention generally relates to the fields of neurology and tissue engineering. More specifically, the present invention relates to the management of aneurysms, specifically intracranial aneurysms, using minimally invasive tissue engineered materials and methods.

### BACKGROUND OF THE INVENTION

As is known in the art, an aneurysm is an abnormal bulging outward of the wall of an artery. In some cases, the bulging may be in the form of a smooth bulge outward in all directions from the artery. This is known as a “fusiform aneurysm.” In other cases, the bulging can be in the form of a sac arising from an arterial branching point or from one side of the artery. This is known as a “saccular aneurysm.”

Aneurysms can occur in any artery of the body, and those occurring in the brain can lead to a stroke. Most saccular aneurysms that occur in the brain comprise a structure in the form of a neck (the opening of which is called “the ostium”), which extends from a cerebral blood vessel and broadens into a pouch projecting away from the vessel.

The problems that manifest as a result of such aneurysms can take several different forms. For example, an aneurysm can form a blood clot within itself. The clot can then break away from the aneurysm and be carried downstream, where it has the potential to obstruct an arterial branch and cause a stroke. Further, the aneurysm can put pressure on surrounding nerves, potentially causing paralysis or abnormal sensation in the facial area or in the adjacent brain. Where pressure is directed to areas of the brain, seizures can result.

If the aneurysm ruptures, blood enters the brain or the subarachnoid space (i.e., the space closely surrounding the brain). This is known as an aneurysmal subarachnoid hemorrhage. In a subarachnoid hemorrhage, the blood floods the subarachnoid space to more or less uniformly coat the brain mass. In a period of about three to four days, the presence of blood components can cause regions of vasospasm, or severe vasoconstriction in which the neural tissue becomes ischemic, resulting in neuronal injury and death. The effects can be severe, covering a large enough portion of the total brain mass to result in serious neurological impairment or death. Of the patients who survive the initial hemorrhage (i.e., 10-15% of patients with the condition die before reaching the hospital for treatment), more than 50% will die within the first thirty days after the hemorrhage. Of those patients who survive, approximately half will suffer a subsequent stroke.

Aneurysmal subarachnoid hemorrhage (“SAH”) is a devastating occurrence affecting approximately 30,000 Americans each year. [1, 2] Though less frequent than other stroke subtypes, SAH affects a younger population and results in 30 to 50% mortality; and 50% significant morbidity in its survivors. [3-6] Even in good clinical grade patients who are expected to have favorable outcomes, when modern quality-of-life outcome measures are applied, there is a significant incidence of depression (36%), poor reintegration into normal living (45%), inability to return to full-time work (33%) and physical disability(23%). [7] Moreover, the economic healthcare costs associated with SAH are enormous. The lifetime economic healthcare cost of an individual in the United States affected by SAH has been calculated to be $228,030, which is the highest among all stroke subtypes. The aggregate lifetime costs for all individuals affected by SAH in the United States in one year have been calculated to be $5.6 billion. [8]

The prevalence of intracranial aneurysms based on prospective autopsy and angiographic studies is estimated to be 3.6 to 6.0% of the population. [9, 10] The predicted risk of SAH from an unruptured intracranial aneurysm is estimated to be 0.05% to 6.0% per year, depending on aneurysm size, aneurysm location, and history of prior subarachnoid hemorrhage from another aneurysm. [11-20]

Given the high fatality attributed to aneurysms and their related complications, the art has addressed treatment of aneurysms using various approaches.

Generally, aneurysms are either treated from outside the blood vessels through surgical techniques or from within the blood vessel using endovascular techniques. Endovascular techniques fall under the broad heading of interventional (i.e., non-surgical) techniques.

Surgical techniques usually involve a craniotomy requiring creation of an opening in the skull of the patient through which the surgeon can insert instruments to operate directly on the brain. In one approach, the brain is retracted to expose the vessels from which the aneurysm arises and then the surgeon places a clip across the neck of the aneurysm, thereby preventing arterial blood from entering the aneurysm. If there is a clot in the aneurysm, the clip also prevents the clot from entering the artery and prevents the occurrence of a stroke. Upon correct placement of the clip, the aneurysm will be obliterated in a matter of minutes.

Surgical techniques are the most common treatment for aneurysms. Unfortunately, surgical techniques for treating these conditions are regarded as major surgery, involving high risk to the patient and necessitating that the patient be of sufficient health and strength to even have a chance of surviving the procedure.

Unfortunately, there are occasions when the size, shape and/or location of an aneurysm make both surgical clipping and endovascular embolization impossible for a particular patient. Generally, the prognosis for such patients is bleak.

As discussed above, endovascular techniques are non-surgical techniques. They are typically performed in an angiography suite using a catheter delivery system. Specifically, known endovascular techniques involve using the catheter delivery system to pack the aneurysm with a material that prevents arterial blood from entering the aneurysm. This technique is generally known as “embolization.”

Recently, endovascular treatment of intracranial aneurysms with platinum-coated electrolytically detachable coils, more commonly referred to as Guglielmi Detachable Coils (“GDC”), has gained worldwide acceptance as a minimally invasive therapeutic alternative to direct surgical methods of craniotomy and microsurgical clip application. The GDC involves intra-aneurysmal occlusion of the aneurysm via a system utilizing a platinum coil attached to a stainless steel delivery wire and electrolytic detachment. Once the platinum coil has been placed in the aneurysm, it is detached from the stainless steel delivery wire by electrolytic dissolution. Specifically, the patient's blood and the saline infusate act as the conductive solutions. The anode is the stainless steel delivery wire and the cathode is a ground needle that is placed in the patient's groin. Once current is transmitted through the stainless steel delivery wire, electrolytic dissolution will occur in the uninsulated section of the stainless steel detachment zone just proximal to the platinum coil (i.e., the platinum coil is unaffected by electrolysis). Other approaches involve the use of materials such as cellulose acetate polymer to fill the aneurysm sac.

Endovascular therapies for intracranial aneurysms have been associated with lower perioperative morbidity and mortality in some studies. [21, 22] The efficacy of endovascular therapies, however, in terms of radio-anatomic occlusion of aneurysms, both immediately and long-term, have not met the high standard previously set by direct surgical methods. [23] Incomplete occlusion and aneurysm remnants occur in as many as 15-85% of aneurysms treated by GDC embolization. [24-27] Aneurysm regrowth or coil compaction after incomplete GDC occlusion occurs in as many as 17%-42% of incompletely occluded small aneurysms and 57-90% of incompletely occluded large aneurysms. [24, 25, 28] There are numerous reports of aneurysm rupture and hemorrhage after incomplete GDC occlusion. [25, 29, 30] One study reports that the annual hemorrhage risk after GDC treatment as high as 2.5% per year. [31]

Thus, while the current endovascular approaches are an advance in the art, they are flawed nonetheless. Specifically, the risks associated with these endovascular approaches include rupturing the aneurysm during the procedure or causing a stroke due to distal embolization of the device or clot from the aneurysm. Additionally, concern exists regarding the long-term potential for endovascular aneurysm obliteration, as there is evidence of intra-aneurysmal rearrangement of the packing material and reappearance of the aneurysm on follow-up angiography.

The problem of aneurysm regrowth or coil compaction after GDC treatment seems to arise from the inability for a neoendothelium to form across the ostium of the treated aneurysm. GDCs occlude aneurysms by promoting thrombosis within the aneurysm sac. [32-39] A number of animal [32, 33, 39, 40] and human autopsy [30, 34, 38, 41-44] studies have revealed that no neoendothelial wall forms across the ostium of aneurysms treated with GDCs, leaving a patent entryway for hemodynamic flow to contribute to aneurysm recanalization and regrowth or coil compaction. Some studies have found a fibrin membrane, but still no evidence of neoendothelialization. [35, 45]

To address this issue, experimental studies have been conducted in animal aneurysm models using coils modified with ion implantation and collagen coating, [46-48] coating with collagen and vascular endothelial growth factor,[49] collagen coating alone, [50, 51] coating with fibroblasts, [52, 53] and coating with bioabsorbable polymeric material. [54]

While some of these studies demonstrated intense scar formation, [48, 51, 54] inflammation, enhanced wall thickness, [49] fibroblast proliferation, [52, 53] and endothelial cell coverage, [48, 50, 51] in all of these studies, modifications were made to platinum coils (GDCs), and whatever tissue or biological reactions took place occurred directly on or in the proximity of the coil surface. Often, the histological changes they found occurred in the sac of the aneurysms, and not in the more critical ostium.

The attempt by these studies to create a biologically-active endovascular treatment which promotes tissue and endothelium formation is a step forward in the appropriate direction. As mentioned, tissue or biological reactions resulting from coil-based treatments occur on or in the proximity of the coil surface, often in the sac, not the ostium of the aneurysm. A tissue-engineered construct, in which the appropriate vascular components are engineered in vitro and incorporated into a bioabsorbable polymer construct, which is suitable for endovascular delivery to the aneurysm and incorporation in vivo, would be highly desirable for inciting neoendothelial formation.

In vitro tissue engineering has been studied as a method of addressing current medical problems such as wound healing and organ transplants. Methods of in vitro tissue engineering involve using harvested cells to populate a carefully prepared scaffold in culture to form an artificial organ. In vitro tissue engineering has enjoyed some success, however, complications such as trauma and healing problems have been associated with such treatments.

Any surgical intervention within a living body is necessarily a traumatic event. The body responds, in most cases, with defense on both acute and chronic time scales. This can be worsened when the surgical intervention implants a foreign object to which the body can have an adverse immune response.

Adverse effects can be tempered through the use of less invasive surgical techniques, such as the endovascular techniques described above, and through the use of autologous cells when performing tissue engineering therapies.

Among the primary objectives of tissue engineering is the integration of the engineered tissue within the patient. This is accomplished by augmenting the cells through the implantation of a supporting device or prosthesis. The replacement of diseased or injured tissue via a suitable transplant developed through such tissue engineering presents a potentially permanent solution.

Accordingly, while advances have been made in the treatment of aneurysms, there is still room for improvement, particularly in approaches to endovascular embolization. Specifically, it would be desirable to have an endovascular prosthesis for use in the embolization of aneurysms that are difficult or impossible to treat otherwise. It would be further desirable if such an endovascular prosthesis could be used to treat aneurysms while mitigating or obviating the disadvantages associated with current endovascular embolization techniques.

### OBJECTS AND SUMMARY OF THE INVENTION

Thus, as stated above, there is a need for tissue engineering-based approaches to aneurysm therapy that involve minimally invasive surgical procedures. Such approaches can provide a scaffold for cellular delivery, such that the cells can multiply and grow, thereby augmenting or replacing the diseased tissue, which will allow the implanted tissue to grow within its “natural” surroundings. The present invention, involving in vivo tissue engineering-based approaches, meets these and other needs.

The present invention is directed to the use of a tissue-engineered construct in which vascular cell components form an intact neoendothelial vascular wall across the aneurysm ostium, preventing any future recanalization or regrowth of the aneurysm. In a preferred embodiment, the construct is comprised of a bioabsorbable polymer coated with autologous vascular cell components. Preferably, the bioabsorbable polymer degrades with time, leaving in place only the incorporated intact neoendothelial vascular wall.

Thus, the present invention comprises compositions, methods and devices for “biologic regeneration” of an intact vascular wall to replace a prior deficiency (e.g., the aneurysm ostium).

This approach is in contrast to other approaches that involve “filling” the aneurysm sac with synthetic materials, metallic or otherwise.

The present invention comprises biological endovascular treatment compositions, devices, and associated methods of use, which are tissue-engineered to form a neoendothelium across the aneurysm (e.g., across the ostium) to provide a permanent cure for intracranial aneurysms. The present invention advantageously provides a minimally invasive approach to the treatment of aneurysms without the problem of aneurysm recanalization or regrowth.

Current methods of treating aneurysms often result in aneurysm recurrence and coil compaction, specifically in regards to treatment of intracranial aneurysms. Histopathologic studies suggest that these defects occur when there is an absence of endothelialization across the aneurysm ostium. Tissue engineered constructs of the present invention advantageously supply vascular endothelium, producing improved therapeutic results. Accordingly, the present invention overcomes defects in the prior art through methods which significantly decrease or eliminate the coil compaction and/or aneurysm recurrence after treatment.

The present invention relates to a method for preventing aneurysm recurrence and/or coil compaction comprising the steps of determining the location of an aneurysm and endovascularly administering to the aneurysm a tissue engineered biopolymer.

The present invention also relates to a method for preventing aneurysm recurrence/and or coil compaction wherein the tissue engineered biopolymer is delivered via a device comprising a double-lumen microcatheter containing two liquid components that polymerize into an elastic gel when combined.

In one embodiment, methods of the present invention advantageously produce a neoendothelium across the ostium of an aneurysm, closing off the aneurysm from the vessel while simultaneously restoring the functionality of the vessel. In comparison to state-of-the-art aneurysm treatments, significantly fewer post-surgical complications and an improved anatomic structure and function of native vessel tissue result.

Other aspects of the invention relate to a device for endovascular administration of the tissue engineered biopolymer.

These and other objects and embodiments are described in or are obvious from the following Detailed Description and are within the scope of the invention.

### BRIEF DESCRIPTION OF THE DRAWINGS

FIG. 1a and FIG. 1b are a schematic and a photograph depicting the location of infusion to the RCAA segment, respectively. FIG. 1c is a schematic depicting the location of the silk ligature tie proximal to the cannulation site of entry.

FIG. 2 is a digital image captured during intravenous digital subtraction angiography depicting a saccular aneurysm at the RCCA stump.

FIG. 3 is a histologic section depicting an aneurysm-parent vessel specimen.

FIG. 4a is a histologic section using Verhoeff's staining to depict the distribution of elastin in the aneurysm-parent specimen. FIG. 4b depicts the enlarged transitional area of the aneurysm-parent specimen. FIG. 4c depicts the enlarged aneurysm vessel wall.

FIG. 5a, FIG. 5b and FIG. 5c are histologic sections stained with hematoxylin-eosin. FIG. 5a depicts the patent right innominate artery with the right common carotid artery occluded. FIG. 5b is a magnified view of the occluded right common carotid artery. FIG. 5c is a further magnified view of the “neck” or origin of the right common carotid artery.

FIG. 6a to 6d depict endothelial cell growth on petri dishes at day 1 FIG. 6a, enlarged at FIG. 6b), day 2 (FIG. 6c) and day 3 (FIG. 6d).

FIG. 6e depicts a histologic section stained with hematoxylin-eosin at day 7.

FIG. 7 depicts a histologic section of a silastic terminus aneurysm aneurysm site stained with hematoxylin-eosin at day 7

### DETAILED DESCRIPTION

In one aspect of the invention there is provided a method of in vivo tissue engineering.

In another aspect of the invention there is provided a method for the in vivo formation of a porous, microcellular scaffold for the attachment, housing and/or growth of autologous, allogenous or xenogenous cells encapsulated therein.

In another aspect of the invention there is provided a method of forming such a scaffold for the in vivo augmentation repair or replacement of diseased, damaged or otherwise compromised tissues of a living body.

In another aspect of the invention there is provided a method of preventing aneurysm recurrence and/or coil compaction comprising determining the location of an aneurysm, endovascularly administering to the aneurysm an effective amount of a first composition comprising a matrix material and a second composition, wherein at least one of the first and second compositions comprise cells, such that the first and second compositions remain separate during the administration, and mixing the first an second compositions at the location of the aneurysm such that a tissue engineered biopolymer is formed, wherein the cells are seeded in the biopolymer scaffold.

Methods of the present invention prevent aneurysm recurrence by regulation of aneurysm formation, growth and/or stability. Preferably, methods of the present invention decrease or eliminate all aneurysm recurrence by decreasing or eliminating aneurysm formation and/or growth and by improving stability. As used herein, the term “aneurysm recurrence” refers to re-growth of a previously existing aneurysm.

Methods of the present invention decrease or eliminate coil compaction by regulating hemodynamics. “Coil compaction” refers to the phenomenon whereby the inserted coils within the aneurysm sac become further compressed after insertion, such that the coils no longer fill the entire volume of the aneurysm. Coil compaction is caused by the arterial pulsatile flow which pushes the coils towards the outer wall or dome of the aneurysm sac and forces the coils to fill a smaller volume of the aneurysm, thereby allowing blood to again flow into at least a portion of the aneurysm, causing instability of the aneurysm.

Preferably, the step of determining the location of an aneurysm can be accomplished by traditional angiography techniques.

Preferably, the methods of the invention can be performed in conjunction with other aneurysm management techniques or additional aneurysm therapies. In one aspect, these additional therapies can comprise detachable coil treatment or other aneurysm packing treatments.

The methods, compositions and devices of the invention can include concurrent or sequential treatment with one or more of enzymes, ions, growth factors, and biologic agents, such as thrombin and calcium or combinations thereof. The methods, compositions and devices of the invention can include concurrent or sequential treatment with non-biologic and/or biologic drugs.

In the efficient attainment of its various aspects, the present invention provides a method of in vivo tissue engineering which mediates tissue healing and regeneration processes by providing, in vivo, a porous, microcellular scaffold. The scaffold is populated by seeded cells or propagating cells from surrounding tissue. Preferably, the cells comprise vascular endothelial cells. A minimally invasive endovascular surgical procedure is utilized for introduction of this system to a vascular site in need of repair in the body.

Preferably, the scaffold is populated by cells through either spontaneous or cellular augmentation, or a combination thereof. In “spontaneous augmentation”, endogenous surrounding cells will migrate and expand in vivo to inhabit the scaffold. “Expand” refers to the replication of a cell or cells. In “cellular augmentation”, exogenous seeded cells are present in the scaffold forming materials when administered to the patient. The term “exogenous” means that the cells originated from outside the organism, were produced outside the organism, or were removed from or modified outside of the organism. The exogenous cells can be derived from a patient's own autologous cell population. The term “autologous” means that the cells occur naturally in the organism. The exogenous cells can also comprise allogenous or xenogenous cells. Allogenous refers to cells that are from a different organism within the same species. Xenogenous refers to cells that are from a different species.

The term “matrix material” refers to a composition which can form a porous, microcellular scaffold or the precursor thereof. As used here in, a matrix material can comprise a liquid or fluidic material having the ability to form a solid or semisolid microcellular scaffold when combined with a suitable catalyst or under suitable conditions.

In another aspect of the invention, the scaffold is comprised of naturally occurring biopolymers. These biopolymers can include fibrin, fibrinogen, fibronectin, collagen, and other suitable biopolymers. Preferably, the scaffold is formed from fibrinogen. The scaffold forming polymers are preferably biodegradable.

In another aspect of the invention, the scaffold can comprise synthetic polymers, including, but not limited to, polyurethanes, polyorthoesters, polyvinyl alcohol, polyamides, polycarbonates, polyvinyl pyrrolidone, marine adhesive proteins, cyanoacrylates, analogs, mixtures, combinations and derivatives of the above.

Biodegradable polyurethanes can be used in this scaffold system. Polyurethanes of the present invention are selected as a result of their structural properties, ease of preparation, and biocompatability.

In yet another aspect of the invention, the scaffold is formed by mixing a first composition comprising a matrix material with a second composition in vivo, such that the scaffold is formed in vivo. The matrix material can comprise fibrin, fibrinogen, fibronectin, collagen, and other suitable biopolymers. In a most preferred aspect of the invention, the matrix material comprises fibrinogen.

In a further aspect of the invention, a second composition can comprise one or more of enzymes, ions, growth factors, and biologic agents.

In a still further aspect of the invention, the second composition comprises one or more catalysts, such as of thrombin, aprotinin and calcium.

In a further preferred aspect of the invention, the composition comprising matrix material further comprises fibroblast growth factor. The fibroblast growth factor is preferably present at a concentration of about 0 to 1000 ng/mL, more preferably, at a concentration of about 100 to 700 ng/mL, still more preferably, at a concentration of about 200 to 400 ng/mL, and further still more preferably, at a concentration of about 250 ng/mL.

In a preferred aspect, the fibrinogen will be obtained from a commercial source or can be collected directly from the patient. The fibrinogen can be purified and processed to form a matrix material. Methods for purifying fibrinogen can include those found in U.S. Pat. No. 5,716,645 and related applications and patents, all of which are incorporated into the present application in their entirety by reference. Specifically, the detailed description and examples of the '645 patent can be useful.

In another aspect of the invention if desired, the first and/or second compositions of the invention can be modified to include non-biologic as well as biologic drugs. The term “non-biologic drugs” encompasses synthetic chemical compounds which are classically referred to as drugs, such as mitomycin C, daunorubicin, and vinblastine, as well as antibiotics.

The biologic drugs that can be added to the first and/or second compositions of the invention include immunomodulators and other biological response modifiers. The term “biological response modifier” is meant to encompass a biomolecule (e.g., peptide, peptide fragment, polysaccharide, lipid, antibody) that is involved in modifying a biological response, such as the immune response or tissue growth and repair, in a manner which enhances a particular desired therapeutic effect, for example, the cytolysis of bacterial cells, the growth of epidermal cells or vasodilation. Biologic drugs can also be incorporated into the fibrinogen compositions of the invention. Those of skill in the art will know, or can readily ascertain, other substances which can act as suitable non-biologic and biologic drugs. For example, the non-biologic or biologic drugs may comprise collagen, fibroblast growth factor, transforming growth factor-beta, endothelial cell growth factor, amicar, and chemotherapeutic agents.

Compositions of the invention can also be modified to incorporate a diagnostic agent, such as a radiopaque agent. The presence of such agents allows the physician to monitor the progression of wound healing occurring internally, i.e. the re-endothelialization of the ostium of an aneurysm. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents which can be utilized in the compositions of the invention can be readily ascertained by those of skill in the art and may include the use of radiolabeled fatty acids or analogs thereof.

The concentration of drug or diagnostic agent in the composition will vary with the nature of the compound, its physiological role, and desired therapeutic or diagnostic effect. The term “therapeutically effective amount” means that the therapeutic agent is present in a sufficient concentration to minimize toxicity, but display the desired effect. Thus, for example, the concentration of an antibiotic where the therapeutic effect is to stimulate the proliferation of endothelial cells at the site of application of the tissue-engineered biopolymer complex would be calculated accordingly. The term “diagnostically effective amount” denotes that concentration of diagnostic agent which is effective in allowing the monitoring of the re-endothelialization, while minimizing potential toxicity. In any event, the desired concentration in a particular instance for a particular compound is readily ascertainable by one of skill in the art.

In yet a further aspect of the invention, the first and second compositions are maintained in liquid form until the time when they are combined. Preferably, the mixing of the first and second compositions creates a gelatinous scaffold. For example, the mixing of the first and second compositions can form a solid-gelatinous polymer upon contact due to the second composition containing a catalyst that promotes cross-linking of the first composition. Further, the scaffold can be formed due to cross-linking of the first and second components upon contact. Yet further still, the formation of the scaffold can occur when the first composition contains a liquid polymer that polymerizes into a solid or gel-like scaffold as a result of the liquid polymer contacting blood. The polymerization can result from an interaction between the liquid polymer and the pH of blood, the ionic composition of blood, or the temperature of blood.

Once the scaffold is generated, it desirably retains its shape and internal architecture to optimize its effectiveness to perform its cell housing function. Advantageously, mixing of the first and second compositions can occur in vivo, upon administration.

Preferably, the first and second compositions are administered to the ostium of an aneurysm.

In still a further aspect of the invention, the first and/or second compostion can be enhanced, or strengthened, through the use of such supplements as human serum albumin (HSA), hydroxyethyl starch, dextran, or combinations thereof. The solubility of the compositions can also be enhanced by the addition of a nondenaturing nonionic detergent, such as polysorbate 80. Suitable concentrations of these compounds for use in the compositions of the invention will be known to those of skill in the art, or can be readily ascertained without undue experimentation. The first and or second compositions can also be further enhanced by the use of optional stabilizers or diluent. The proper use of these would be known to one of skill in the art, or can be readily ascertained without undue experimentation.

In a further aspect of the invention, the present invention desirably employs tissue engineering techniques which allow the tissue-engineered biopolymer to be seeded with cells. This seeding can occur after the scaffold has been formed in vivo, or it can take place prior to the scaffold formation. In a preferred aspect, the cells can be introduced into the first and or second composition prior to administration, such that they are present when the compositions are administered to the patient, and the scaffolding is formed. Alternatively, the cells can be administered in a step separate from the administration of the first and second compositions.

In a preferred embodiment, the cells are present in one or more of the first and or second composition at a concentration of about 1×10⁸ cells/mL to 1×10³ cells/mL, more preferable from about of about 1×10⁷ cells/mL to 1×10⁴ cells/mL, and still more preferably from about 1×10⁶ cells/mL to 1×10⁵ cells/mL.

Techniques for cell seeding tissue engineered constructs include those described in U.S. Pat. No. 6,455,311, the contents of which are herein incorporated by reference.

In a most preferred aspect of the invention, the cells are autologous cells. Preferably, these cells will be harvested from the patient and cultured prior to introduction into one or more of the first or second compositions. The cells will be selected based on function. In a preferred aspect, vascular and endothelial cells will be collected and cultured in order to create a neoendothelium across the opening of the ostium. Additionally, stem cells can also be utilized.

In one embodiment, the endothelial cells and/or the vascular cells are derived from vascular tissue, preferably pulmonary artery, pulmonary vein, femoral artery, femoral vein, saphenous artery, saphenous vein, iliac artery, iliac vein, umbilical artery, umbilical vein, microvascular tissue, adipose, placental, and aortic tissue. Microvascular tissue is preferably derived from heart, lung, liver, kidney, brain or dermal tissue, and can be autologous or heterologous to a subject who will receive the tissue-engineered scaffolding or device of the present invention. Isolation of endothelial cells are exemplified by work by Jaffe and coworkers [55]. The identity of endothelial cells can be confirmed by their production of von Willebrand factor (vWF); and uptake of acylated low-density lipoprotein (acLDL). Harvesting and isolation of smooth muscle cells are described in Ross [56]. Vascular smooth muscle cells can be advantageously identified by the presence of α-actin, desmin, and smooth muscle myosin. Antibodies against these smooth muscle cell-specific cellular markers are well known in the art and are commercially available.

In a preferred embodiment, the endothelial cells and/or the vascular cells are derived from stem cells. The stem cells can be embryonic stem (ES) cells [57], embryonic germ (EG) cells, multipotent adult progenitor cells (MAPCs) [58], hematopoietic stem cells (HSCs) [59] (See also Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al, U.S. Pat. No. 5,716,827), mesenchymal stem cells (MSCs) [60], and endothelial progenitor cells (EPCs) [61, 62]. Stem cells can be derived from any appropriate tissue, and are preferably derived from bone marrow, brain, spinal cord, umbilical cord blood, liver, placenta, blood, adipose tissue, or muscle. The stem cells from which endothelial and/or vascular cells are differentiated can be autologous or heterologous to a subject who will receive the tissue-engineered scaffold or device.

In vivo, cells surrounding the scaffold can enter the scaffold through cell migration. In this aspect, the cells surrounding the scaffold can be attracted by biologically active materials, including biological response modifiers, such as polysaccharides, proteins, peptides, genes, antigens and antibodies which are selectively incorporated into the scaffold to provide the needed selectivity, for example, to tether the cell receptors to the scaffold or stimulate cell migration into the scaffold, or both. The scaffold is porous, having interconnecting channels that allow for cell migration, augmented by both biological and physical-chemical gradients.

In a preferred embodiment, cells surrounding the scaffold can be attracted by biologically active materials including one ore more of VEGF, fibroblast growth factor, transforming growth factor-beta, endothelial cell growth factor, P-selectin, and intercellular adhesion molecule. One of skill in the art will recognize and know how to use other biologically active materials that are appropriate for attracting cells to the scaffold.

In a preferred embodiment, biomolecules are incorporated into the scaffold forming materials, causing the biomolecules to be imbedded within the scaffold. Alternatively, chemical modification methods may be used to covalently link a biomolecule on the surface of the scaffold. The surface functional groups of the scaffold components can be coupled with reactive functional groups of the biomolecules to form covalent bonds using coupling agents well known in the art such as aldehyde compounds, carbodiimides, and the like. Additionally, a spacer molecule may be used to gap the surface reactive groups in collagen and the reactive groups of the biomolecules to allow more flexibility of such molecules on the surface of the scaffold. Other similar methods of attaching biomoleucules to the interior or exterior of a scaffold will be known to one of skill in the art.

Preferably, the scaffold and cellular assembly are either fully or partially implanted into the surrounding tissue to become a functioning part thereof. Preferably, the implant initially attaches to and communicates with the host through a cellular monolayer.

Preferably, over time, the seeded cells will expand and migrate out of the biopolymer scaffold to the surrounding tissue. More preferably, the cells will form a confluent layer on the surface of the cellular wall. Still more preferably, the confluent cell layer is integrated into the existing cell wall. Even more preferably, the confluent cell layer forms a neoendothelium across the ostium.

In one aspect of the invention, cells that are placed in one or more of the first and second compositions can optionally be encapsulated into a biodegradable, protective capsule comprising a membrane, the surface of which is modified with tethers that attach the enclosure to the scaffold. The capsule preferably contains nutrients for the cells to survive and propagate. Each encapsulated cell expands into a functional unit. Microencapsulation technology is frequently used to encapsulate living cells, as illustrated in U.S. Pat. No. 5,084,350, hereby incorporated by reference.

In another aspect of the invention, there is provided a method of increasing endothelialization across an aneurysm ostium comprising determining the location of an aneurysm, endovascularly administering to the aneurysm an effective amount of a first composition comprising a matrix material and a second composition, wherein at least one of the first and second compositions comprise cells, such that the first and second compositions remain separate during the administration; and mixing the first and second compositions at the location of the aneurysm such that a tissue engineered biopolymer scaffold is formed, wherein the cells are seeded in the biopolymer scaffold.

In another aspect of the invention there is provided a method of producing a neoendothelium comprising determining the location of an aneurysm, endovascularly administering to the aneurysm an effective amount of a first composition comprising a matrix material and a second composition, wherein at least one of the first and second compositions comprises cells, such that the first and second compositions remain separate during the administration; and mixing the first and second compositions at the location of the aneurysm such that a tissue engineered biopolymer scaffold is formed, wherein the cells are seeded in the biopolymer scaffold.

In a further aspect of the invention, an endovascular device is provided which will allow for endovascular delivery of the first and second compositions, such that the compositions are separated one from the other until administration to the site of the aneurysm.

In a still further aspect of the invention, the device is a microcatheter which contains dual cannulae for simultaneous administration of two separate compositions.

In the efficient attainment of its various aspects, the present invention provides a method of in vivo tissue engineering which mediates tissue healing and regeneration processes by providing, in vivo, a coated aneurysm maintenance device. The device can comprise any synthetic structure suitable for intravascular administration. A minimally invasive endovascular surgical procedure is utilized for introduction of the device to a vascular site to be repaired in the body.

The coating of the aneurysm maintenance device can comprise any of the biopolymers or other related substances previously described, including fibrin, fibrinogen, hydrogels, etc. In a further aspect of the invention, the coating forms a scaffold on the outer surface of the device.

Preferably, the scaffold is seeded with cells and/or biologically active materials, as previously described. In one aspect of the invention, the biologically active materials attract endogenous cell migration into the scaffold.

In yet a further aspect of the invention, the traditional aneurysm maintenance device comprises coils, stents, or other related devices.

Preferably, the device is inserted endovascularly into the aneurysm sac or across the ostium of the aneurysm, in accordance with traditional placement of a standard aneurysm maintenance device. Preferably, the seeded cells and/or the migrating endogenous cells create a neoepithelium to fill the aneurysm, as in the case of aneurysm coils, or to close off the ostium of the aneurysm, as in a stent.

In a further aspect of the invention there is provided a method of preventing aneurysm recurrence and or coil compaction comprising determining the location of an aneurysm, endovascularly administering to the aneurysm a traditional aneurysm maintenance device, wherein the device is coated with a biocompatible material, wherein cells are seeded in the biocompatible material.

Preferably, the biocompatible material comprises fibrin, fibrinogen, fibronectin, collagen, and other suitable biopolymers, hydrogels, vicryl suture, Tisseel and other suitable polymers.

The device can comprise biodegradable polyurethanes, which are selected as a result of their structural properties, the ease of their preparation, and their biocompatability.

The aneurysm maintenance device is populated by seeded cells or propagating cells from surrounding tissue. Tissue engineering techniques can be employed to seed the tissue-engineered biopolymer coating of the aneurysm maintenance device with cells. This seeding can occur after the aneurysm maintenance device has been implanted, or it can take place prior to the implantation. In a preferred embodiment, the cells are present in a concentration of about 1×10⁸ cells/mL to 1×10³ cells/mL, more preferable from about of about 1×10⁷ cells/mL to 1×10⁴ cells/mL, and still more preferably from about 1×10⁶ cells/mL to 1×10⁵ cells/mL.

Preferably, the step of determining the location of an aneurysm can be accomplished by traditional angiography techniques (e.g., detection of barium sulfate as well as various organic compounds containing iodine, such as iocetamic acid, iodipamide, iodoxamate meglumine, iopanoic acid, as well as diatrizoate derivatives, such as diatrizoate sodium. Other contrast agents which can be utilized in the compositions of the invention can be readily ascertained by those of skill in the art).

### EXAMPLES

The invention will now be further described by the following non-limiting examples. One of skill in the art would recognize the utility of, and would possess the ability to practice, alternative methods and variations from those described herein.

### Example 1

### In Vitro Use of Tissue-Engineered Biopolymer

Porcine and ovine endothelial cells were harvested from pig and sheep carotid arteries using a collagenase cell isolation technique which has been previously described [63].

Fibrin biopolymer (Baxter, Glendale, Calif.) was tissue engineered with fibroblast growth factor (250 ng/mL) and living endothelial cells (porcine, 2.2×10⁶ cells/mL; ovine, 1.5×10⁶ cells/mL) (TEFBP).

TEFBP was delivered via a microcatheter onto a 100 cm petri dish and maintained in medium at 37° C. for 24 hours, after which a section of the TEFBP was explanted onto a new petri dish with the same conditions and monitored for endothelial cell growth. The explant step was performed to prove that aberrant endothelial cells were not delivered to the petri dish separate from the TEFBP.

The petri dishes were examined at 24 hour intervals for seven days to determine the extent of endothelial growth.

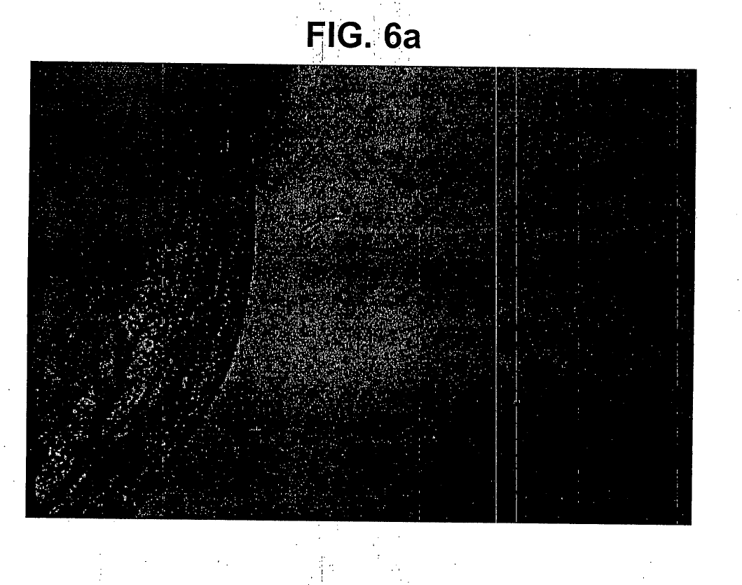

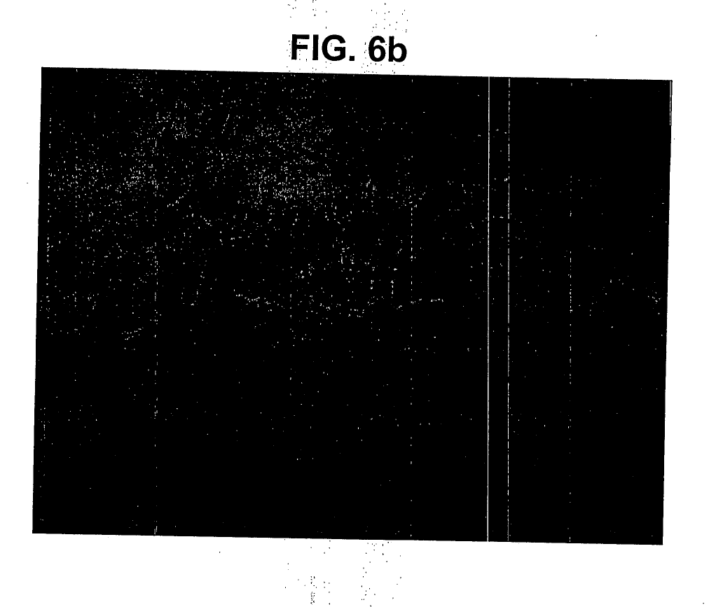

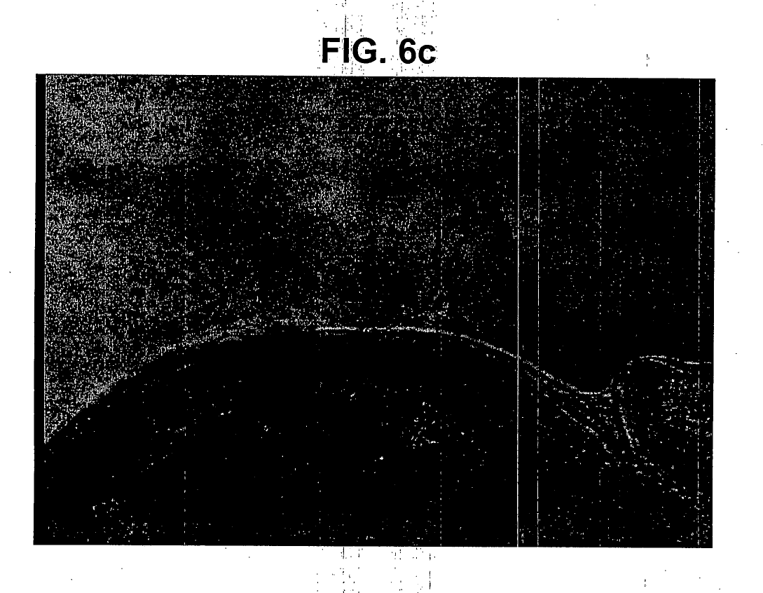

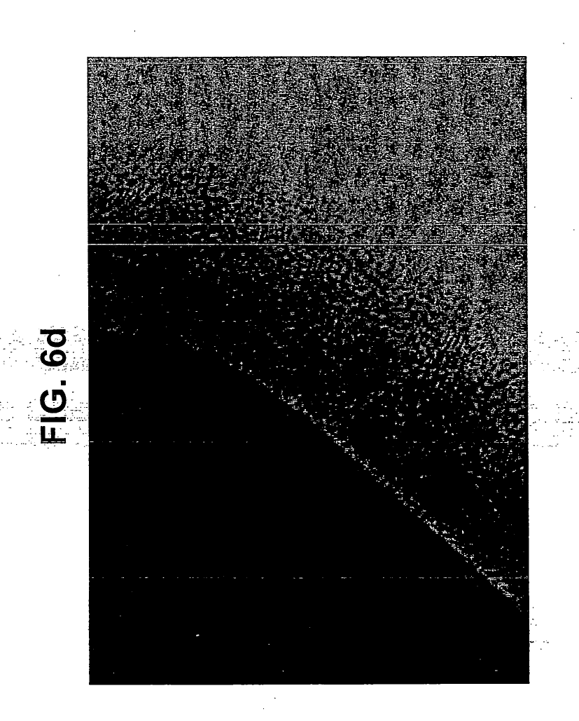

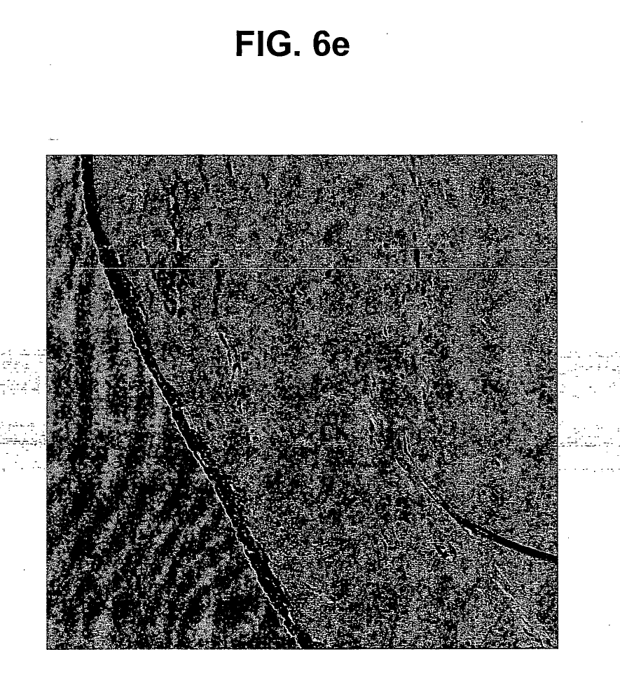

Examination of the petri dishes at 24 hours showed endothelial cell growth within the TEFBP but not beyond the surface (FIGS. 6a and 6b). At 48 hours, there was endothelial cell growth on the surface of the TEFBP and spreading out onto the surface of the petri dish (FIG. 6c). At 72 hours, there was a confluent cell layer covering the entire surface area of the petri dish (FIG. 6d). After 7 days, TEFBP was extracted and fixed with 10% formalin and embedded in parafm for staining with hematoxylin-eosin. Hematoxylin-eosin stains of the TEFBP after 7 days showed sporadic endothelial cell growth within the TEFBP but a complete intact endothelial cell layer on the surface (FIG. 6e).

Clearly, endothelial cells were able to proliferate and migrate out of the TEFBP scaffolding to create the intact endothelial cell layer.

### Example 2

### Use of TEFBP in Silastic Terminus Aneurysm Model

Porcine and ovine endothelial cells were harvested as previously described, namely, porcine and ovine endothelial cells were harvested from pig and sheep carotid arteries using a collagenase cell isolation technique our group has previously described [63].

Fibrin biopolymer (Baxter, Glendale, Calif.) was tissue engineered with fibroblast growth factor (250 ng/mL) and living endothelial cells (porcine, 2.2×10⁶ cells/mL; ovine, 1.5×10⁶cells/mL) as mentioned above.

TEFBP was delivered via a microcatheter into a silastic terminus aneurysm model and was then subjected to flow conditions of medium at 37° C. being pumped at 96 systolic pulsations/minute for 7 days. After day 7, the TEFBP was extracted, fixed with 10% formalin, and embedded in parafin for staining with hematoxylin-eosin.

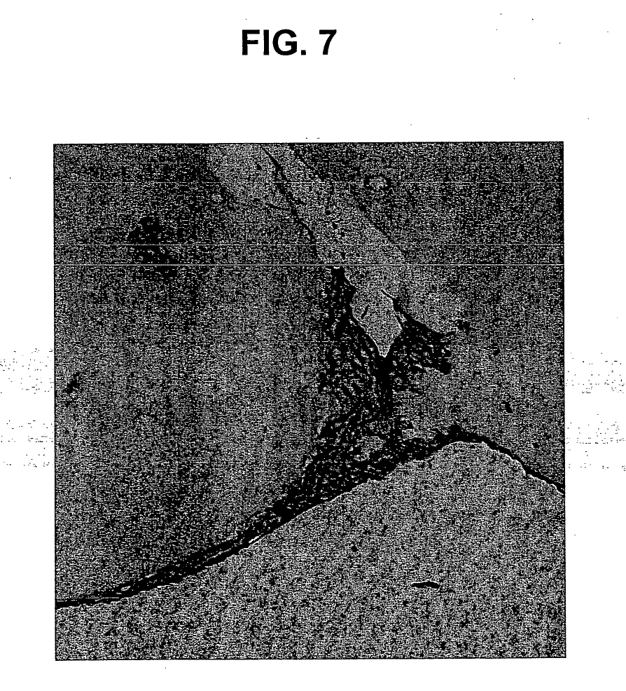

Hematoxylin-eosin stains of the TEFBP extracted from the silastic terminus aneurysm showed only sporadic endothelial cell growth within the TEFBP, but a complete intact endothelial cell layer had formed across the ostium of the aneurysm (FIG. 7).

### Example 3

### In Vitro Use of Tissue-Engineered Biopolymer

A bioabsorbable tissue-engineered polymer will be designed with autologuous vascular cell components. This will be done in a similar manner as that which has been previously described for cardiovascular bioprosthetic structures in Dover lambs [64-67] [68, 69], however, this design will be for use with New Zealand white rabbits.

Endovascular deployment devices will be designed to deliver the polymer construct to aneurysms intravascularly. A previously described, well-established experimental rabbit aneurysm model [70] will be used, wherein a right common carotid artery stump is surgically created and incubated endoluminally with Elastase (Porcine; Sigma, St. Louis and Worthington Biochemical, Lakewood, N.J.). Four weeks later, a saccular aneurysm will be found which closely resembles human intracranial aneurysms in histology, hemodynamics, morphology, size, blood pressure, hematology, and coagulation [70, 71].

Transfemoral angiography will then be performed to demonstrate the radiographic morphology of the aneurysm. During the same angiographic procedure, the tissue-engineered polymer will be deployed endovasculary to the aneurysm (control animals will be treated with endovascularly-delivered Guglielmi detachable coils or sham treatment).

At various timepoints (2 weeks, 3 months, 6 months), the animals will then undergo repeat angiography to demonstrate whether the aneurysms are still radiographically occluded, or whether aneurysm recanalization, regrowth, or coil compaction has occured. Immediately following angiography, the animals will be euthanized and the aortic arch and brachiocephalic vessels will be removed en bloc.

The explanted vessels will be evaluated histologically by hematoxylin and eosin stain for gross morphology, by environmental scanning electron microscopy, a Movat pentachrome stain for extracellular matrix components, and immunohistochemical staining with human von Willebrand factor for the identification of endothelial cells. Biochemical quantification assays will be performed to determine DNA content [72], collagen content [73], elastin [72], proteoglycan-glycosaminoglycan content [74], metalloproteinases (MMPs) and tissue inhibitors of MMPs [75, 76], and protein content [75], to compare to native vessel.

It is expected that the explanted vessels will show that the bioabsorbable polymer has started to degrade with time, leaving in place only the incorporated, intact neoendothelial vascular wall. This will demonstrate that the tissue engineered biopolymer construct acts to incite formation of a neoendothelium across the aneurysm ostium, and will provide a permanent cure for intracranial aneurysms by a minimally invasive approach without the problem of aneurysm recanalization or regrowth.

### Example 3.1

### Alternate Rabbit Aneurysm Model

It is important to have a reliable experimental animal model for aneurysms which is histologically and hemodynamically accurate and simulates true human intracranial aneurysms. Evaluation of therapies for their biological effects on processes such as scar formation and endothelialization necessitates an experimental aneurysm model which mimics the true histology of real human intracranial aneurysms. Likewise, the experimental aneurysm model should simulate the shear stresses and hemodynamic forces which are significant factors in coil compaction and aneurysm recurrence in true human intracranial aneurysms treated with coil embolization.

A modified approach to a previously described elastase aneurysm model [70], [77, 78] (see example 3) was applied to create saccular aneurysms. This model is characterized by histology and/or hemodynamics that are similar to true human aneurysms.

All animal experimentation was performed in accordance with a protocol approved by the Subcommittee on Research Animal Care. Procedures were performed in 14 female New Zealand white rabbits weighing 3.0-4.0 kg. Of the 14 rabbits, 12 underwent the aneurysm creation procedure, and a control procedure was performed in 2 animals.

Prior to all procedures, sufficient anesthesia was induced with a single administration of intramuscular ketamine 60 mg/kg, xylazine 6 mg/kg, and acepromazine 1 mg/kg. Intubation or mechanical ventilation was not necessary in any of the animals.

After induction of anesthesia, fur was shaved, skin was prepared with betadine solution, and sterile drapes were used. An 8-10 cm midline incision was made from the thyroid cartilage to the sternum. The right sternomastoid muscle and the right sternohyoid muscle were dissected and retracted laterally revealing the trachea and the deep neck muscles. Working to the right of the trachea, dissection and retraction of the right sternothyroid muscle laterally revealed the underlying RCCA. The insertion of the right sternomastoid muscle to the sternum was lifted. The attachments of the right pectoralis tenuis muscle to the top of the sternum were reflected. This revealed the origin of the RCCA and the right subclavian artery (RSCA) from the brachiocephalic trunk underlying or just rostral to the insertion of the first rib to the sternum. In all 14 animals the origin of the right common carotid artery was visualized without having to remove the first rib. In a few animals, the thymus gland was overlying the origin of the right common cartoid artery and was reflected.

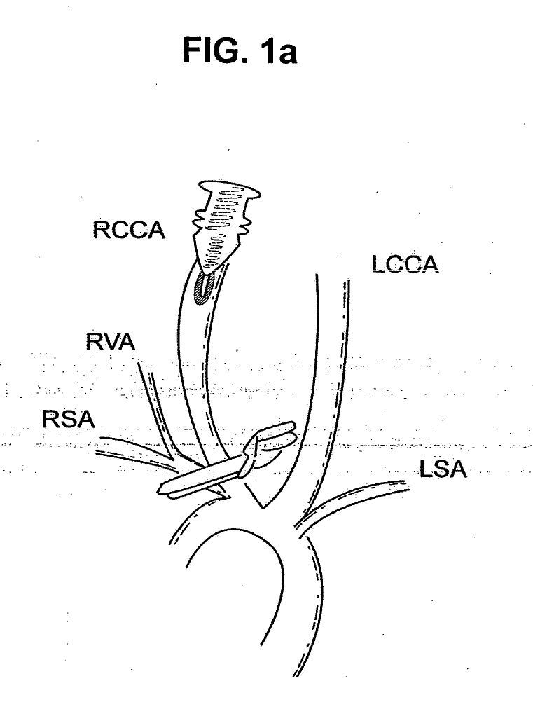

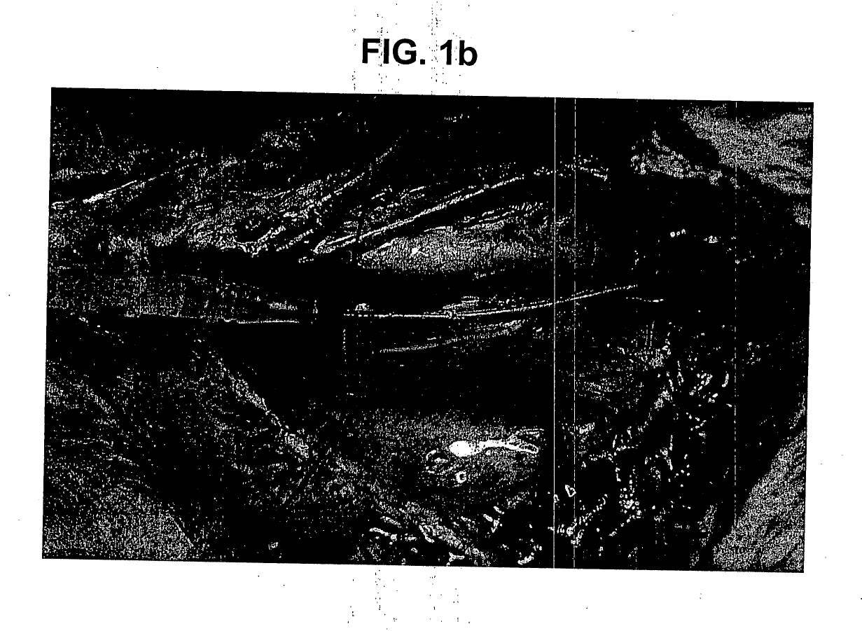

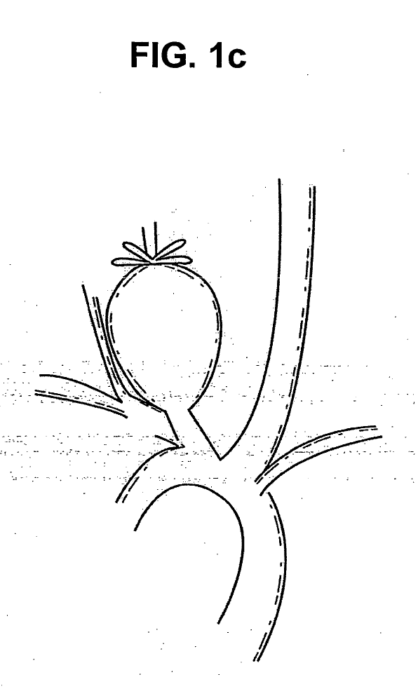

A 3-0 silk ligature was used to occlude the RCCA distally creating a stump. A temporary aneurysm clip was then placed at the origin of the RCCA. A 24-guage angiocatheter was used to cannulate the RCCA stump in a retrograde fashion from distal-to-proximal. Remaining blood within the isolated RCCA segment was aspirated, and 100 units porcine pancreatic elastase (Worthington Biochemical Corp., Lakewood, N.J.) were infused into the lumen of the isolated RCCA segment and allowed to incubate for 20 minutes (FIGS. 1a and 1b). Supplemental elastase was infused if elastase leaked out. After 20 minutes, a 3-0 silk ligature was tied just proximal to the cannulation site of entry of the angiocatheter and the temporary aneurysm clip was released (FIG. 1c).

The skin was reapproximated with a running subcuticular 4-0 undyed vicryl suture.

In the control procedure, the RCCA was surgically exposed in the same manner as the aneurysm creation procedure. An RCCA stump was created by a distal 3-0 silk ligature, however, no temporary clip was placed and the stump was not cannulated by an angiocatheter. The skin was reapproximated with a running subcuticular 4-0 undyed vicryl suture.

Animals were assessed neurologically on post-procedure days 1, 2, 3, 7, 14, and 21 using a rabbit neurologic grading scale and food intake scale previously described [79]. All 14 rabbits (12 experimental, 2 control) had Grade I neurologic assessments and Grade I food intake assessments on post-procedure days 1, 2, 3, 7, 14, and 21. There were no mortalities.

Intravenous digital subtraction angiography (IVDSA) was performed in animals 21 days after their aneurysm creation procedure or control procedure. The animal was anesthetized with a single administration of intramuscular ketamine 60 mg/kg, xylazine 6 mg/kg, and acepromazine 1 mg/kg. The left or right marginal ear vein was cannulated with a 24-guage angiocatheter. Five milliliters of nonionic, iodinated low-osmolar contrast (Ultravist, Berlex Laboratories, Inc., Wayne and Montville, N.J.) was injected intravenously and digital subtracted images were filmed at a rate of two frames per second in the arterial phase.

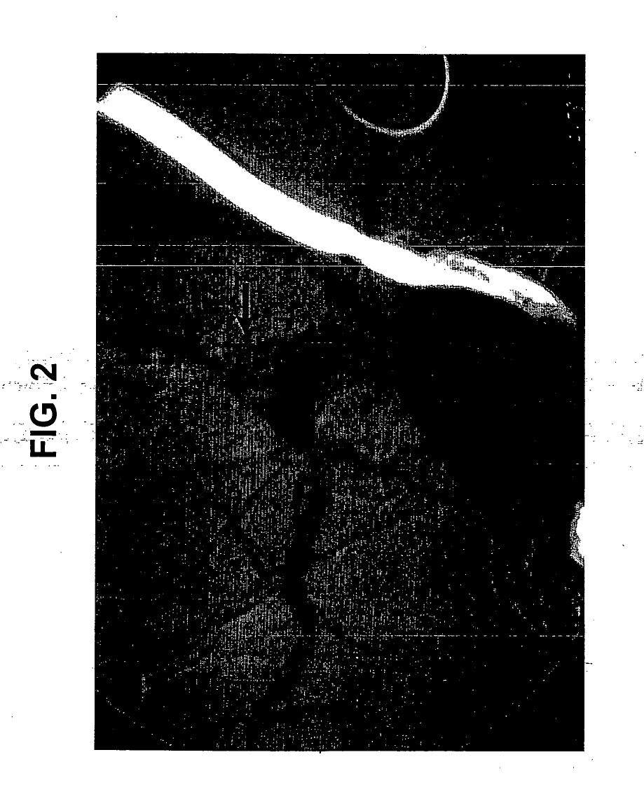

All 12 rabbits that underwent aneurysm creation procedures demonstrated saccular aneurysms at the RCCA stump on IVDSA performed on post-procedure day 21 (FIG. 2). Mean aneurysm size was 5.9±1.9 mm (mean±standard deviation). Aneurysm size range was 4.3-10.8 mm. The close proximity of the LCCA to the origin of the RCCA on the aortic arch of the New Zealand white rabbit closely resembles a bifurcation aneurysm.

Both rabbits that underwent control procedures showed no aneurysm and complete occlusion of the RCCA stump on IVDSA on post-procedure day 21.

Animals were euthanized by rapid intravenous injection of high-dose sodium thiopental. The right brachiocephalic trunk and RCCA aneurysm were harvested, flushed with saline, and fixed in 10% formaldehyde for more than 24 hours. Specimens were embedded in paraffin, sectioned, and stained with hematoxylin-eosin (H&E) and Verhoeff's stain (with eosin counterstaining to differentiate the tissue background).

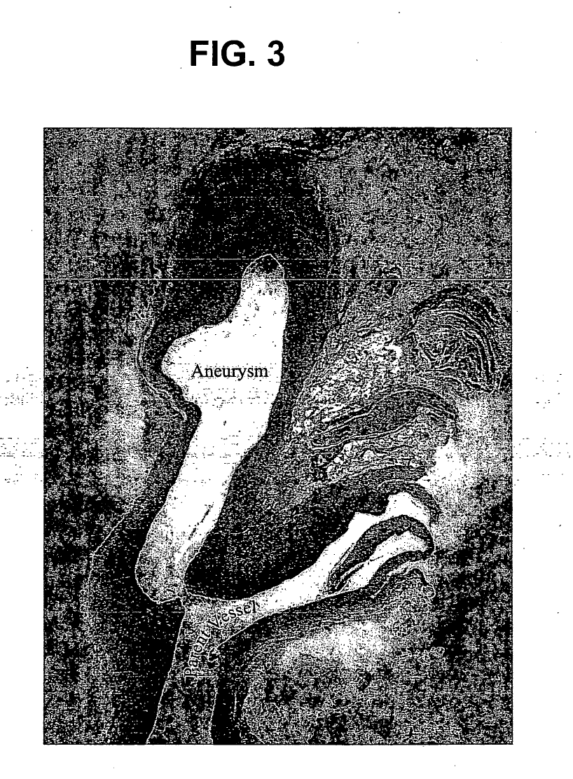

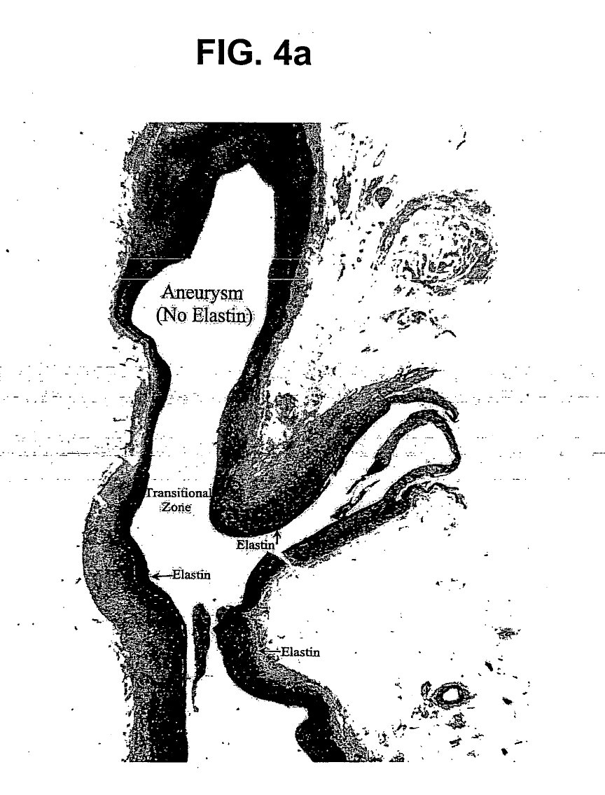

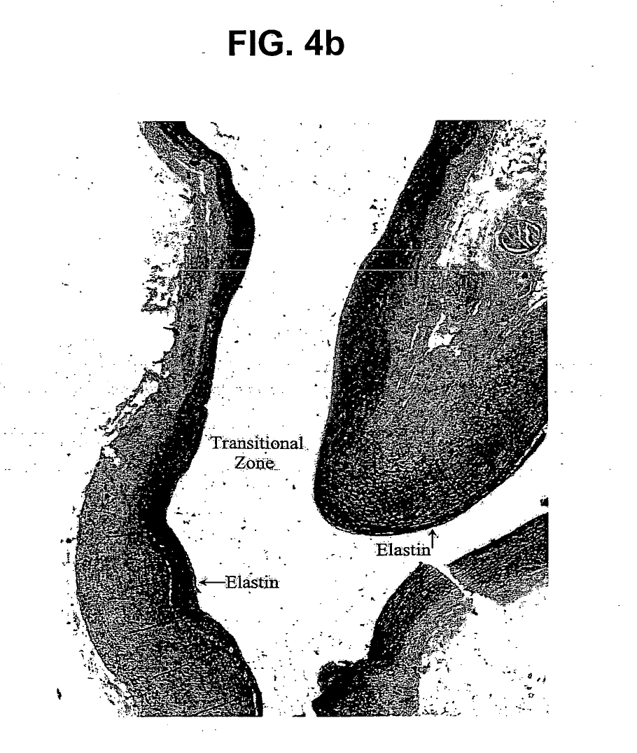

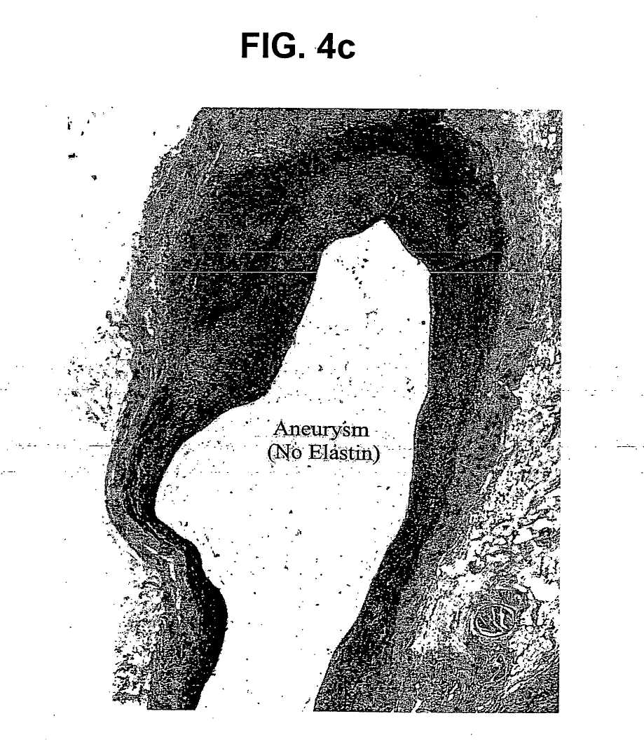

H&E staining of aneurysm-parent vessel specimens harvested from euthanized experimental animals demonstrated normal parent vessel wall (brachiocephalic trunk and right subclavian artery) and abnormal vessel wall of the aneurysm (FIG. 3). The aneurysm wall demonstrated abnormal thinning, loss of normal vessel wall elements, absence of an inflammatory reaction, and marked loss of cellular elements in the sac wall and absence of fibromuscular neointimal proliferation (FIG. 4a). Verhoeff's staining (stains for elastin) of aneurysm-parent vessel specimens harvested from euthanized experimental animals with eosin counterstain demonstrated no elastin in the aneurysm, a transitional zone at the ostium, and intact elastin in the parent vessel (FIG. 4b).

### Example 4

### Methylcellulose Polymer Seeded with Endothelial Cells

Sheep endothelial cells were harvested from sheep carotid artery by a collagenase cell isolation technique previously described [63].

The sheep endothelial cells were suspended at a concentration of 5-6×10⁶ in a 1 mL suspension (DMEM with FBS, penicillin and glutamine) which was resuspended in 1 mL methylcellulose polymer.

The methylcellulose polymer and cell suspension was then placed in the center of a 100 cm petri plate and placed in a 37° C. incubator for 10 minutes and allowed to harden. 10 mL of medium was added (DMEM with FBS, penicillin and glutamine) and the plate was returned to a 37° C. incubator.

At 24, 48, and 72 hours after incubation, microscopic examination was performed to analyze endothelial cell growth. At 24 hours, live endothelial cells were visible within the polymer. At 48 hours, cell migration had occurred on the surface of the polymer and cell spreading out onto the plate was also visible. At 72 hours, a confluent cell layer was visible.

At 7 days post-incubation, the polymer was fixed in 10% formalin for H & E staining.

This experiment demonstrated that a heat-activated polymer such as methylcellulose, which polymerizes at 37° C., can be seeded with endothelial cells. In in vitro experiments, at 48-72 hours, the endothelial cells migrated to the surface of the polymer and grew outward, forming a confluent layer. This would be ideal for endovascular treatment of human aneurysms, during which the cell-seeded polymer can be delivered as a liquid via a microcatheter, and will polymerize when it is deposited into the aneurysm due to the natural body temperature (37° C.). The endothelial cells would then migrate outward and multiply, forming a confluent layer across the aneurysm ostium.

### Example 5

### Guglielmi Detachable Coils Coated with Fibrin Sealant and Seeded with Endothelial Cells

Sheep endothelial cells were harvested from sheep carotid artery by a collagenase cell isolation technique previously described [63]. The sheep endothelial cells were suspended at a concentration of 5-6×10⁶ in 30 mL DMEM with FBS, penicillin and glutamine.

A Guglielmi detachable coil was coated with fibrin sealant (Tisseel, Baxter, Glendale, Calif.) and placed in the cell suspension in a Falcon tube which was slowly rotated in a 37° C. incubator for 48 hours. After the 48 hour seeding process, the coil was placed in the center of a 100 cm petri plate and 10 mL of medium was added (DMEM with FBS, penicillin and glutamine) before the plate was placed in a 37° C. incubator.

Endothelial cell growth was monitored by microscopic examination at 24, 48, and 72 hours. Microscopic examination at 24 hours did not show endothelial cell migration. However, at 48 hours, there was cell migration spreading out from the coil onto the plate and at 72 hours, there was a confluent cell layer present.

At 7 days, the coil was fixed in 10% formalin for analysis by electron misroscopy.

This example demonstrated that detachable coils can be modified to contain cells by coating the coils with fibrin sealant and seeding with endothelial cells. In vitro experiments demonstrate that at 48-72 hours the endothelial cells migrated out from the coil and continued to grow outwards, forming a confluent layer. This is ideal for endovascular treatment of human aneurysms, because detachable coils are widely used for the treatment of human aneurysms and have an efficient delivery system into the sac of human aneurysms. This modification of the coils would allow endothelial cells to migrate outwards and multiply, forming a confluent layer across the aneurysm ostium.

### Example 6

### Guglielmi Detachable Coils Coated with Methylcellulose Polymer and Seeded with Endothelial Cells

Sheep endothelial cells were harvested from sheep carotid artery by a collagenase cell isolation technique previously described [63]. The sheep endothelial cells were suspended at a concentration of 5-6×10⁶ in 30 mL DMEM with FBS, penicillin and glutamine.

A Guglielmi detachable coil was coated with methylcellulose polymer and placed in the cell suspension in a Falcon tube which was slowly rotated in a 37° C. incubator for 48 hours. After the 48 hour seeding process, the coil was placed in the center of a 100 cm petri plate and 10 mL of medium was added (DMEM with FBS, penicillin and glutamine) before the plate was placed in a 37° C. incubator.

Endothelial cell growth was monitored by microscopic examination at 24 hours, 48 hours, and 72 hours. Microscopic examination at 24 hours did not show endothelial cell migration. However, at 48 hours, there was cell migration spreading out from the coil onto the plate and at 72 hours, there was a confluent cell layer.

At day 7, the coil was fixed in 10% formalin for analysis by electron microscopy.

This demonstrates that detachable coils can be modified by coating with methylcellulose polymer and seeding with endothelial cells. In vitro experiments conclude that by 48-72 hours, the endothelial cells migrate out from the coil and continue to grow outwards, forming a confluent layer. This is ideal for endovascular treatment of human aneurysms because detachable coils are widely used for the treatment of human aneurysms and have an efficient delivery system into the sac of human aneurysms. Modification of the coils with methylcellulose polymer allows endothelial cells to migrate out of the coil and multiply, forming a confluent layer across the aneurysm ostium.

### Example 7

### Vicryl Suture Seeded with Endothelial Cells

Sheep endothelial cells were harvested from sheep carotid artery by a collagenase cell isolation technique previously described [63] The sheep endothelial cells were suspended at a concentration of 5-6×10⁶ in 30 mL DMEM with FBS, penicillin and glutamine.

A vicryl suture was placed in the cell suspension in a Falcon tube which was slowly rotated in a 37° C. incubator for 48 hours. After the 48 hour seeding process, the suture was placed in the center of a 100 cm petri plate and 10 mL of medium was added (DMEM with FBS, penicillin and glutamine) before the plate was placed in a 37° C. incubator.

Endothelial cell growth was monitored by microscopic examination at 24 hours, 48 hours, and 72 hours. Microscopic examination at 24 hours did not show endothelial cell migration. However, at 48 hours, there was evidence of cell migration spreading out from the suture onto the plate, and at 72 hours, there was a confluent cell layer present.

At day 7, the suture was fixed in 10% formalin for analysis by electron microscopy.

Vicryl sutures represent a bioabsorbable material that is known to cause a natural wound healing process and which can be modified or incorporated into a coil or other such scaffold that can easily be deliverable via a microcather to fill the sac of an aneurysm endovascularly. As shown here, vicryl can be seeded with endothelial cells. In vitro experiments at 48-72 hours demonstrate that endothelial cells migrate out from the vicryl and continue to grow outwards, forming a confluent layer. This is ideal for endovascular treatment of human aneurysms, because the cell-seeded vicryl can be easily delivered into the sac of human aneurysms via a microcatheter, and the endothelial cells will then migrate out from the bioabsorbable vicryl and multiply, forming a confluent layer across the aneurysm ostium.

### Example 8

### Cell-Seeded Fibrin Glue for Aneurysm Management

As described in Example 3.1, elastase model aneurysms are created in New Zealand white rabbits. Autologous venous endothelial cells are then harvested and cultured from the jugular veins of the same rabbit using a collagenase method described by Conte et al [80, 81].

When a sufficient population of endothelial cells has grown, Tisseel fibrin glue (Baxter) is then seeded with the endothelial cells as previously described [82, 83], and is injected into the rabbits' elastase aneurysms. Seven days later, angiography is performed prior to euthanizing the rabbits and harvesting the aneurysms with the parent artery vessel. Histological analysis is then performed to examine whether an endothelium has grown. Control rabbits will have their elastase aneurysms 1) treated with Guglielmi detachable coils; 2) treated with Tisseel without endothelial cells; 3) untreated.

### Example 9

### Cell-Seeded Aneurysm Management Devices

As described in Example 3.1 and Example 8, elastase model aneurysms are created in New Zealand white rabbits. Autologous venous endothelial cells are then harvested and cultured from the jugular veins of the same rabbit using a collagenase method described by Conte et al [80, 81].

As shown in Example 5, aneurysm management devices such as coils will be coated with a biocompatible material. The biocompatible material can be fibrin glue as discussed in Example 5, or can comprise comprises fibrin, fibrinogen, fibronectin, collagen, and other suitable biopolymers, hydrogels, vicryl suture, Tisseel and other suitable polymers. Cultured cells will then be seeded onto the coated aneurysm management devices. These devices can include Guglielmi detachable coils, Matrix coils, Hydrocoil coils, Onyx, Neuroform stents, and nanofibers. The aneurysm management devices will then be endovascularly inserted at the site of the aneurysm.

The cell-seeding of the coated devices would allow endothelial cells to migrate outwards and multiply, forming a confluent layer across the aneurysm ostium.

### Example 10

### In Vivo Use of Cell-Seed Fibrin Glue

Autologous venous endothelial cells were harvested and cultured from the jugular veins of the same rabbit using a collagenase method described by Conte et al [80, 81]. When a sufficient population of endothelial cells had grown, Tisseel fibrin glue (Baxter) was seeded with the endothelial cells as previously described [82, 83].

The cell seeded Tisseel fibrin glue was then injected into the right common carotid artery of rabbits. 7 days later, the right common carotid artery (with the right innominate and right subclavian artery) was harvested and histopathology was performed on the sample. Specifically, specimens were embedded in paraffin, sectioned, and stained with hematoxylin-eosin (H&E).

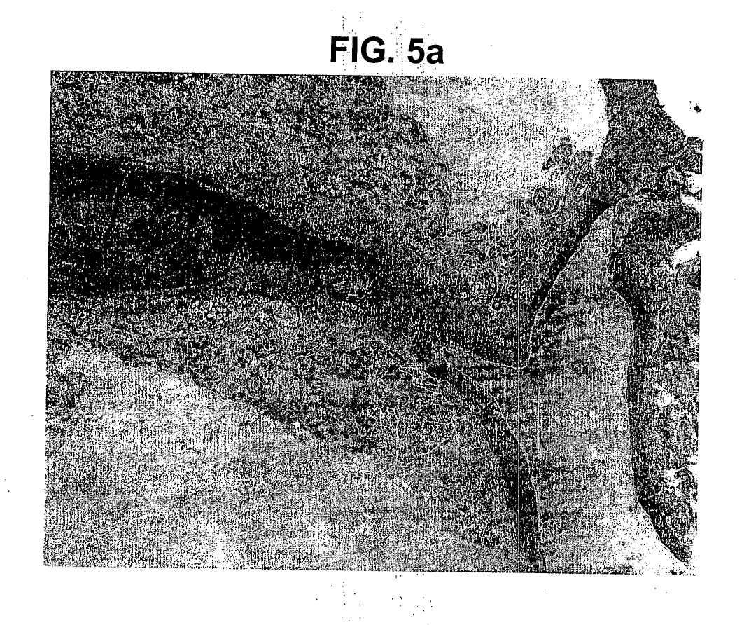

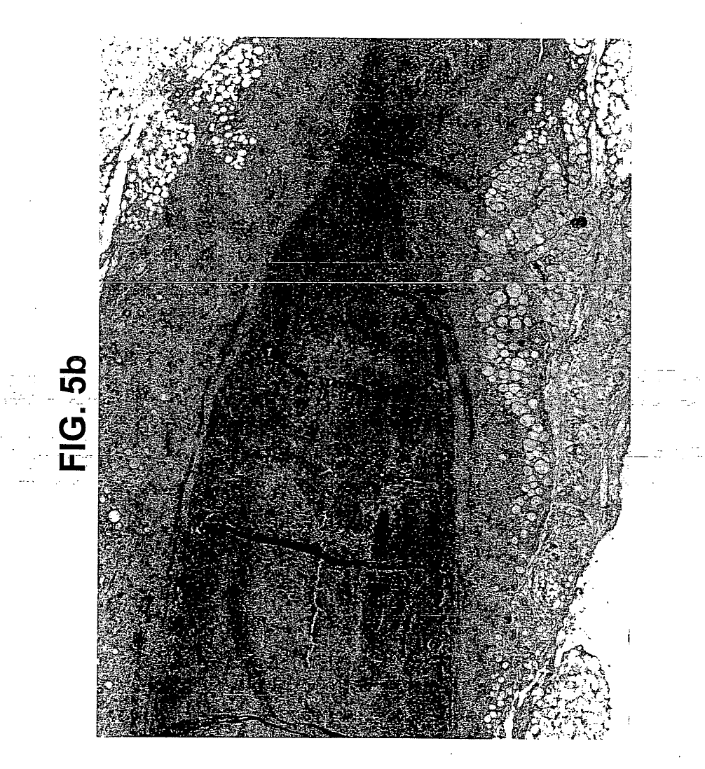

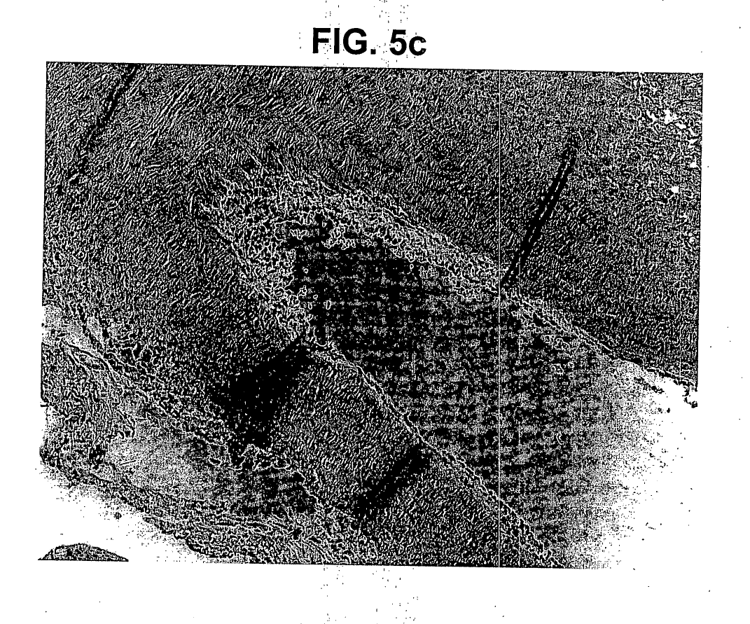

As seen in FIGS. 5a-c, after 7 days the fibrin glue completely occluded the lumen of the right common carotid artery, endothelial cells were growing within the fibrin glue, and an intact layer of endothelium was present across the “neck” or origin of the right common carotid artery. FIG. 5a shows the patent right innominate artery with the right common carotid artery filled with Tisseel and endothelial cells. FIG. 5b shows the present of endothelial cells in the Tisseel. FIG. 5c shows the endothelial cell layer across the “neck” or origin of the right common carotid artery.

### REFERENCES

1. Detailed Diagnoses and Procedures, National Hospital Discharge Survey, 1990, ed. U.D.o.H.a.H. Services. Vol. PHS 92-1774. Series 13. 1992, Hyattsville, Md.: DHHS publication.
2. Mayberg M R, et al., Guidelines for the management of aneurysmal subarachnoid hemorrhage. A statement for healthcare professionals from a special writing group of the stroke council, American Heart Association. Stroke, 1994. 25(11): p. 2315-2328.
3. Torner J C, Epidemiology of subarachnoid hemorrhage. Semin Neurol, 1984. 4: p. 354-369.
4. Ostergaard J R, Risk factors in intracranial saccular aneurysms. Aspects on the formation and rupture of aneurysms, and development of cerebral vasospasm. Acta Neurol Scand, 1989. 80: p. 81-98.
5. Longstreth W J, et al., Clinical course of spontaneous subarachnoid hemorrhage: a population-based study in King County, Washington. Neurology, 1993. 43: p. 712-718.
6. Ingall T J and Wiebers D O, Natural history of subarachnoid hemorrhage, in Stroke: Populations, Cohorts, and Clinical Trials, Whisnant J P, Editor. 1993, Butterworth-Heinemann Ltd: Boston, Mass.
7. Carter B S, et al., Factors associated with reintegration to normal living after subarachnoid hemorrhage. Neurosurgery, 2000. 46(6): p. 1326-1334.
8. Taylor T N, et al., Lifetime cost of stroke in the United States. Stroke, 1996. 27(9): p. 1459-1466.
9. Rinkel G J, Dijibuti M, and van Gijn J, Prevalence and risk of rupture of intracranial aneurysms: a systematic review. Stroke, 1998. 29: p. 251-256.
10. Wardlaw J M and White P M, The detection and management of unruptured intracranial aneurysms. Brain, 2000. 123(2): p. 205-221.
11. ISUIA Investigators, Unruptured intracranial aneurysms: risks of rupture and risks of surgical intervention. N Engl J Med, 1998. 339: p. 1725-1733.
12. Inagawa T, Hada H, and Katoh Y, Unruptured intracranial aneurysms in elderly patients. Surg Neurol, 1992. 38: p. 364-370.
13. Asari S and Ohmoto T, Natural history and risk factors of unruptured cerebral aneurysms. Clin Neurol Neurosurg, 1993. 95: p. 205-214.
14. Yasui N, et al., Long-term followup study of unruptured intracranial aneurysms. Neurosurgery, 1997. 40: p. 1155-1159.
15. Yasui N, et al., Subarachnoid hemorrhage caused by previously diagnosed, previously unruptured intracranial aneurysms: a retrospective analysis of 25 cases. Neurosurgery, 1996. 39: p. 1096-1100.
16. Wiebers D O, et al., The significance of unruptured intracranial saccular aneurysms. J Neurosurg, 1987. 66: p. 23-29.
17. Wiebers D O, Whisnant J P, and O'Fallon W M, The natural history of unruptured intracranial aneurysms. N Engl J Med, 1981. 304: p. 696-698.
18. Juvela S, Porras M, and Heiskanen O, Natural history of unruptured intracranial aneurysms: a long-term follow-up study. J Neurosurg, 1993. 79: p. 174-182.
19. Winn H R, et al., The long-term outcome in patients with multiple aneurysms: incidence of late hemorrhage and implications for treatment of incidental aneurysms. J Neurosurg, 1983. 59: p. 642-651.
20. Heiskanen O, Risk of bleeding from unruptured aneurysm in cases with multiple intracranial aneurysms. J Neurosurg, 1981. 55: p. 524-526.
21. Vanninen R, et al., Ruptured intracranial aneurysms: acute endovacular treatment with electrolytically detachable coils. A prospective randomized study. Radiology, 1999. 211: p. 306-308.
22. Gruber D P, et al., A comparison between endovascular and surgical management of basilar artery apex aneurysms. J Neurosurg, 1999. 90(5): p. 868-874.
23. Hoh B L, et al., Results after surgical and endovascular treatment of paraclinoid aneurysms by a combined neurovascular team. Neurosurgery, 2001. 48(1): p. 78-89.
24. Hope J K, Byrne J V, and Molyneux A J, Factors influencing successful angiographic occlusion of aneurysms treated by coil embolization. AJNR Am J Neuroradiol, 1999. 20(3): p. 391-399.
25. Bavinzski G, et al., Treatment of basilar artery bifurcation aneurysms by using Guglielmi detachable coils: a 6-year experience. J Neurosurg, 1999. 90(5): p. 843-852.
26. Vinuela F, Duckwiler G, and Mawad M, Guglielmi detachable coil embolization of acute intracranial aneurysms: perioperative anatomical and clinical outcome in 403 patients. J Neurosurg, 1997. 86(3): p. 475-482.
27. Fernandez Zubillaga A, et al., Endovascular occlusion of intracranial aneurysms with electrically detachable coils: correlation of aneurysm neck size and treatment results. AJNR Am J Neuroradiol, 1994. 15(5): p. 815-820.
28. Hayakawa M, et al., Natural history of the neck remnant of a cerebral aneurysm treated with the Guglielmi detachable coil system. J Neurosurg, 2000. 93(4): p. 561-568.
29. Tateshima S. et al., Endovascular treatment of basilar tip aneurysms using Guglielmi detachable coils: anatomic and clinical outcomes in 73 patients from a single institution. Neurosurgery, 2000. 47(6): p. 1332-1339.
30. Manabe H, et al., Rerupture of coil-embolized aneurysm during long-term observation. Case report. J Neurosurg, 1998. 88: p. 1096-1098.
31. Gruber A, et al., Clinical and angiographic results of endosaccular coiling treatment of giant and very large intracranial aneurysms: a 7-year, single center experience. Neurosurgery, 1999. 45(4): p. 793-803.
32. Macdonald R L, et al., Randomized comparison of Guglielmi detachable coils and cellulose acetate polymer for treatment of aneurysms in dogs. Stroke, 1998. 29: p. 478-486.
33. Reul J, et al., Long-term angiographic and histopathologic findings in experimental aneurysms of the carotid bifurcation embolized with platinum and tungsten coils. AJNR Am J Neuroradiol, 1997. 18: p. 35-42.
34. Bavinzski G, et al., Gross and microscopic histopathologic findings in aneurysms of the human brain treated with Guglielmi detachable coils. J Neurosurg, 1999. 91: p. 284-293.
35. Horowitz M B, et al., Scanning electron microscopic findings in a basilar tip aneurysm embolized with detachable coils. AJNR Am J Neuroradiol, 1997. 18: p. 688-690.
36. Koizumi T, et al., Histological finding in aneurysm treated with interlocking detachable coils: scanning electron microscopical study. Neurol Surg, 1997. 25: p. 1027-1031.
37. Reul J, et al., Endovascular occlusion of experimental aneurysms with detachable coils: influence of packing density and perioperative anticoagulation. Neurosurgery, 1997. 41: p. 1160-1168.
38. Shimizu S, et al., Tissue response of a small saccular aneurysm after incomplete occlusion with a Guglielmi detachable coil. AJNR Am J Neuroradiol, 1999. 20: p. 546-548.
39. Kallmes D F, et al., Histologic evaluation of platinum coil embolization in an aneurysm model in rabbits. Radiology, 1999. 213: p. 217-222.
40. Spetzger U, et al., Microsurgically produced bifurcation aneurysms in a rabbit model for endovascular coil embolization. J Neurosurg, 1996. 85: p. 488-495.
41. Molyneux A J, et al., Histological findings in giant aneurysms treated with Guglielmi detachable coils. Report of two cases with autopsy correlation. J Neurosurg, 1995. 83: p. 129-132.
42. Gaebel C, et al., Pathology of human intracranial aneurysms after GDC-embolization and correlation with animal model data. Clin Neuropathol, 1996. 15: p. 270.
43. Mizoi K, et al., A pitfall in the surgery of a recurrent aneurysm after coil embolization and its histological observation: technical case report. Neurosurgery, 1996. 39: p. 165-169.
44. Manabe H, et al., Embolisation of ruptured cerebral aneurysm with interlocking detachable coils in actue stage. Acute IDC embolisation for ruptured aneurysms. Intervent Neuroradiol, 1997. 3: p. 49-63.
45. Stiver S I, et al., Acute human histopathology of an intracranial aneurysm treated using Guglielmi detachable coils: case report and review of the literature. Neurosurgery, 1998. 43(5): p. 1203-1208.
46. Murayama Y, et al., Ion implantation and protein coating of detachable coils for endovascular treatment of aneurysms: concepts and preliminary results in swine models. Neurosurgery, 1997. 40(6): p. 1233-1244.
47. Murayama Y, et al., Development of a biologically active Guglielmi detachable coil for the treatment of cerebral aneurysms. Part I: in vitro study. AJNR Am J Neuroradiol, 1999. 20: p. 1986-1991.
48. Murayama Y, et al., Development of the biologically active Guglielmi detachable coil for the treatment of cerebral aneurysms. Part II: an experimental study in a swine aneurysm model. AJNR Am J Neuroradiol, 1999. 20: p. 1992-1999.
49. Abrahams J M, et al., Delivery of human vascular endothelial growth factor with platinum coils enhances wall thickening and coil impregnation in a rat aneurysm model. AJNR Am J Neuroradiol, 2001. 22: p. 1410-1417.
50. Tamatani S, et al., Radiologic and histopathologic evaluation of canine artery occlusion after collagen-coated platinum microcoil delivery. AJNR Am J Neuroradiol, 1999. 20: p. 541-545.
51. Dawson R C, et al., Treatment of experimental aneurysms using collagen-coated microcoils. Neurosurgery, 1995. 36(1): p. 133-140.
52. Kallmes D F, et al., Platinum coil-mediated implantation of growth factor-secreting endovascular tissue grafts: an in vivo study. Radiology, 1998. 207: p. 519-523.
53. Marx W F, et al., Endovascular treatment of experimental aneurysms by use of biologically modified embolic devices: coil-mediated intraaneurysmal delivery of fibroblast tissue allografts. AJNR Am J Neuroradiol, 2001. 22: p. 323-333.
54. Murayama Y, et al., Bioabsorbable polymeric material coils for embolization of intracranial aneurysms: a preliminary experimental study. J Neurosurg, 2001. 94: p. 454-463.
55. Jaffe, E. A., et al., Culture of human endothelial cells derived from umbilical veins. Identification by morphologic and immunologic criteria. J. Clin. Invest., 1973. 52(11): p. 2745-2756.
56. Ross, R., The smoth muscle cell. II. Growth of smooth muscle in culture and formation of elastic fibers. J. Cell Biol., 1971. 50(1): p. 172-186.
57. Lebkowski, J. S., et al., Human embryonic stem cells: culture, differentiation, and genetic modification for regenerative medicine applications. Cancer J., 2001. 7(Supp 2): p. S83-S93.
58. Jiang, Y., et al., Pluripotency of mesenchymal stem cells derived from adult marrow. Nature, 2002. 418: p. 41-49.
59. Hill, B., et al., High-level expression of a novel epitope of CD59 identifies a subset of CD34+ bone marrow cells highly enriched for pluripotent stem cells. Exp. Hematol., 1996. 8: p. 936-943.
60. Fridenshtein, A., Stromal bone marrow cells and the hematopoietic microenvironment. Arkh. Pathol., 1982. 44: p. 3-11.
61. Asahara, T., et al., Isolation of putative progenitor endothelial cells for angiogenesis. Science, 1997. 275: p. 964-967.
62. Kubo, H. and K. Alitalo, The bloody fate of endothelial stem cells. Genes Dev., 2003. 17: p. 322-329.
63. Piper, H., ed. Cell culture techniques in heart and vessel research. 1990, Springer-Verlag: New York.
64. Sodian R, et al., Evaluation of biodegradable, three-dimensional matrices for tissue engineering heart valves. ASAIO Journal, 2000. 46: p. 107-110.
65. Hoerstrup S P, et al., Functional living trileaflet heart valves grown in vitro. Circulation, 2000. 102(suppl III): p. III-22-III-49.
66. Sodian R, et al., Early in vivo experience with tissue-engineered trileaflet heart valves. Circulation, 2000. 102(suppl III): p. III-22-III-29.
67. Stock U A, et al., Dynamics of extracellular matric production and turnover in tissue engineered cardiovascular structures. Journal of Cellular Biochemistry, 2001. 81: p. 220-228.
68. Stock U A, et al., Tissue-engineered valved conduits in the pulmonary circulation. J Thorac Cardiovasc Surg, 2000. 119: p. 732-740.
69. Stock U A, et al., Patch augmentation of the pulmonary artery with bioabsorbable polymers and autologous cell seeding. J Thorac Cardiovasc Surg, 2000. 120: p. 1158-1168.
70. Altes T A, et al., Creation of saccular aneurysms in the rabbit: a model suitable for testing endovascular devices. AJR Am J Roentgenol, 2000. 174(2): p. 349-354.
71. Abruzzo T, et al., Histologic and morphologic comparison of experimental aneurysms with human intracranial aneurysms. AJNR Am J Neuroradiol, 1998. 19: p. 1309-1314.
72. Kim B S, et al., Optimizing seeding and culture methods to engineer smooth muscle tissue on biodegradable polymer matrices. Biotechnol Bioeng, 1998. 57: p. 46-54.
73. Bergman I and Loxley R, Two improved and simplified methods for the spectrophotometric determination of hydroxyproline. Anal Chem, 1963. 35: p. 1961-1965.
74. Carney S L, Proteoglycans, in Carbohydrate analysis: a practical approach, Chaplin M F and Kennedy J F, Editors. 1987, IRL Press: Oxford. p. 97-141.
75. Moses M A and Shing Y, Production of matrix metalloproteinases and a metalloproteinase inhibitor by swarm rat chondrosarcoma. Biochem Biophys Res Commun, 1994. 199: p. 418-424.
76. Peters C A, et al., Dysregulated proteolytic balance as the basis of excess extracellular matrix in fibrotic disease. Am J Physiol, 1997. 272: p. R1960-1965.
77. Fujiwara, N., et al., Serial angiography in an elastase-induced aneurysm model in rabbits: evidence for progressibe aneurysm enlargement after creation. AJNR Am J Neuroradiol, 2001. 22: p. 698-703.
78. Short J G, et al., Elastase-induced saccular aneurysms in rabbits: comparison of geometric features with those of human aneurysms. AJNR Am J Neuroradiol, 2001. 22: p. 1833-1837.
79. Endo S, Branson P J, and Alksne J F, Experimental model of symptomatic vasospasm in rabbits. Stroke, 1988. 19: p. 1420-1425.
80. Conte, M., et al., Endothelial seeding fails to attenuate intimal thickening in ballon-injured rabbit arteries. J Vasc Surg, 1995. 21(3): p. 413-421.
81. Conte, M., et al., Efficient repopulation of denuded rabbit arteries with autologous genetically modified endothelial cells. Circulation, 1994. 89(5): p. 2161-2169.
82. Hoh, B., et al., Tissue-engineered fibrin biopolymer as a novel endovascularly-deliverable treatment for intracranial aneurysms: in vitro study. Journal of Neurosurgery, 2003. 98: p. A240-A241.
83. Hoh, B., et al. Tissue-engineered fibrin biopolymer as a novel endovascularly-deliverable treatment for intracranial aneurysms: in vitro study. in Sixth Annual Meeting of the AANS/CNS Section on Cerebrovascular Surgey and the American Society of Interventional and Therapeutic Neuroradiology. 2003. Phoenix, Ariz.

1. A method of regulating aneurysm formation, growth and/or stability comprising:
identifying an aneurysm;
endovascularly administering to the aneurysm an effective amount of a first composition comprising a matrix material and a second composition, such that the first and second compositions remain separate during administration and wherein at least one of the first and second compositions comprise cells;
mixing the first and second compositions at the location of the aneurysm to form a polymer scaffold comprising said cells.

2. The method of claim 1, wherein the endovascular administration is performed via one or more microcatheters.

3. The method of claim 1, wherein the scaffold is placed across an ostium of the aneurysm.

4. The method of claim 1, wherein the cells expand and migrate out of the scaffold to the surrounding tissue.

5. The method of claim 1, wherein the cells are autologous cells.

6. The method of claim 5, wherein the cells are selected from the group consisting of vascular cells, endothelial cells and stem cells.

7. The method of claim 6, wherein the cells are endothelial cells.

8. The method of claim 4, wherein the cells form a confluent layer.

9. The method of claim 8, wherein the confluent cell layer is integrated into the existing cell wall.

10. The method of claim 9, wherein the integrated, confluent cell layer forms a neoendothelium across the ostium.

11. The method of claim 1, wherein the matrix material comprises one or more materials selected from the following: fibrin, fibrinogen, collagen, polyorthoesters, polyvinyl alcohol, polyamides, polycarbonates, polyvinyl pyrrolidone, marine adhesive proteins, cyanoacrylates, analogs, mixtures, combinations and derivatives of the above.

12. The method of claim 11, wherein the matrix material comprises fibrinogen.

13. The method of claim 1, wherein the second composition comprises one or more of enzymes, ions, growth factors, and biologic agents.

14. The method of claim 13, wherein the second composition comprises thrombin and calcium.

15. The method of claim 1, wherein the method is performed in conjunction with at least one additional therapy for treating aneurysms.

16. The method of claim 15, wherein the additional therapy comprises an aneurysm packing treatment.

17. A method of increasing endothelialization across an aneurysm ostium comprising:
identifying an aneurysm;
endovascularly administering to the aneurysm an effective amount of a first composition comprising a matrix material and a second composition, such that the first and second compositions remain separate during the administration and wherein at least one of the first and second compositions comprise cells;
mixing the first and second compositions at the location of the aneurysm to form a polymer scaffold comprising said cells.

18. The method of claim 17, wherein the endovascular administration is performed via one or more microcatheters.

19. The method of claim 17, wherein the scaffold is placed across an ostium of the aneurysm.

20. The method of claim 17, wherein the cells expand and migrate out of the scaffold to the surrounding tissue.

21. The method of claim 17, wherein the cells are autologous cells.

22. The method of claim 21, wherein the cells are selected from the group consisting of vascular cells, endothelial cells and stem cells.

23. The method of claim 22, wherein the cells are endothelial cells.

24. The method of claim 20, wherein the cells form a confluent layer.

25. The method of claim 24, wherein the confluent cell layer is integrated into the existing cell wall.

26. The method of claim 25, wherein the integrated, confluent cell layer forms a neoendothelium across the ostium.

27. The method of claim 17, wherein first matrix material comprises one or more matrix materials selected from the following: fibrin, fibrinogen, collagen, polyorthoesters, polyvinyl alcohol, polyamides, polycarbonates, polyvinyl pyrrolidone, marine adhesive proteins, cyanoacrylates, analogs, mixtures, combinations and derivatives of the above.

28. The method of claim 27, wherein matrix material comprises fibrinogen.

29. The method of claim 17, wherein the second composition comprises one or more of enzymes, ions, growth factors, and biologic agents.

30. The method of claim 29, wherein the second composition comprises thrombin and calcium.

31. The method of claim 17, wherein the method is performed in conjunction with at least one additional therapy for treating aneurysms.

32. The method of claim 31, wherein the additional therapy is an aneurysm packing treatment.

33. A method of producing a neoendothelium comprising:
identifying an aneurysm;
endovascularly administering to the aneurysm an effective amount of a first composition comprising a matrix material and a second composition, such that the first and second compositions remain separate during the administration and wherein at least one of the first and second compositions comprise cells, and
mixing the first and second compositions at the location of the aneurysm to form a polymer scaffold comprising said cells.

34. The method of claim 33, wherein the endovascular administration is performed via one or more microcatheters.

35. The method of claim 33, wherein the scaffold is placed across an ostium of the aneurysm.

36. The method of claim 33, wherein the cells expand and migrate out of the polymer scaffold to the surrounding tissue.

37. The method of claim 33, wherein the cells are autologous cells.

38. The method of claim 37, wherein the cells are selected from the group consisting of vascular cells, endothelial cells and stem cells.

39. The method of claim 38, wherein the cells are endothelial cells.

40. The method of claim 36, wherein the cells form a confluent layer.

41. The method of claim 40, wherein the confluent cell layer is integrated into the existing cell wall.

42. The method of claim 41, wherein the integrated, confluent cell layer forms a neoendothelium across the ostium.

43. The method of claim 33, wherein the matrix material comprises one or more matrix materials selected from the following: fibrin, fibrinogen, collagen, polyorthoesters, polyvinyl alcohol, polyamides, polycarbonates, polyvinyl pyrrolidone, marine adhesive proteins, cyanoacrylates, analogs, mixtures, combinations and derivatives of the above.

44. The method of claim 43, wherein the matrix material comprises fibrinogen.

45. The method of claim 33, wherein the second composition comprises one or more of enzymes, ions, growth factors, and biologic agents.

46. The method of claim 45, wherein the second composition comprises thrombin and calcium.

47. The method of claim 33, wherein the method is performed in conjunction with at least one additional therapy for treating aneurysms.

48. The method of claim 47, wherein the additional therapy comprises an aneurysm packing treatment.

49. An endovascular device comprising a microcatheter that comprises two cannulae, wherein one cannula contains a composition comprising a matrix material and all other compositions are contained within the second cannula, such that the composition comprising the matrix material remains separate until administration.

50. The endovascular device of claim 49, wherein the first and second compositions are delivered simultaneously.

51. A method of increasing endothelialization across an aneurysm ostium comprising:
identifying an aneurysm;
endovascularly administering to the aneurysm an aneurysm maintenance device, wherein the device is coated with a biocompatible material comprising cells;
and forming a polymer scaffold comprising said cells.

52. The method of claim 51, wherein the endovascular administration is performed via one or more microcatheters.

53. The method of claim 51, wherein the cells expand and migrate out of the biocompatible material to the surrounding tissue.

54. The method of claim 51, wherein the cells are autologous cells.

55. The method of claim 54, wherein the cells are selected from the group consisting of vascular cells, endothelial cells and stem cells.

56. The method of claim 55, wherein the cells are endothelial cells.

57. The method of claim 53, wherein the cells form a confluent layer.

58. The method of claim 57, wherein the confluent cell layer is integrated into the existing cell wall.

59. The method of claim 58, wherein the integrated, confluent cell layer forms a neoendothelium across the ostium.

60. The method of claim 51, wherein the biocompatible material comprises one or more matrix materials selected from the following: fibrin, fibrinogen, collagen, polyorthoesters, polyvinyl alcohol, polyamides, polycarbonates, polyvinyl pyrrolidone, marine adhesive proteins, cyanoacrylates, hydrogels, vicryl suture, Tisseel, and analogs, mixtures, combinations and derivatives of the above.

61. The method of claim 60, wherein the biocompatible material additionally comprises one or more of enzymes, ions, growth factors, and biologic agents.

62. A method of decreasing or eliminating coil compaction comprising:
identifying an aneurysm;
endovascularly administering to the aneurysm an effective amount of a first composition comprising a matrix material and a second composition, such that the first and second compositions remain separate during administration and wherein at least one of the first and second compositions comprise cells;
mixing the first and second compositions at the location of the aneurysm to form a polymer scaffold comprising said cells.
